(19) United States
(12) Patent Application Publication
(10) Pub. No.: US 2009/0208540 A1
(43) Pub. Date: Aug. 20, 2009

(54) IMPLANTABLE DEVICE FOR THE DELIVERY OF NALTREXONE AND METHODS OF USE THEREOF

(75) Inventors: Petr Kuzma, Princeton, NJ (US); Harry Quandt, Bensalem, NJ (US); Sheng-hung Kuo, Plainsboro, NJ (US)

Correspondence Address: FOLEY AND LARDNER LLP;SUITE 500, 3000 K STREET NW, WASHINGTON, DC, 20007, US

(73) Assignee: Indevus Pharmaceuticals, Inc.

(21) Appl. No.: 12/351,617

(22) Filed: Jan. 9, 2009

Related U.S. Application Data
(63) Continuation-in-part of application No. 12/242,497, filed on Sep. 30, 2008.
Application No. 12/242,497 is a continuation-in-part of application No. 10/915,625, filed on Aug. 10, 2004.
(60) Provisional application No. 60/494,132, filed on Aug. 11, 2003.

(51) Int. Cl. A61K 9/00; A61K 31/439

(52) U.S. Cl. 424/400; 514/282

(57) ABSTRACT

This invention is related to the use of polyurethane-based polymer as a drug delivery device to deliver naltrexone and formulations thereof at a constant rate for an extended period of time. The devices and methods of use thereof are biocompatible and biostable, and useful for the delivery of naltrexone in patients (humans and animals).

### RELATED APPLICATIONS

This application is a Continuation-In-Part of U.S. application Ser. No. 12/242,497, filed on Sep. 30, 2008, which is a Continuation-In-Part of U.S. application Ser. No. 10/915,625, filed Aug. 10, 2004, which claims the benefit of U.S. Provisional Application 60/494,132, filed Aug. 11, 2003, the entire contents of each of which are hereby incorporated herein by reference.

### BACKGROUND

Due to its excellent biocompatibility, biostability and physical properties, polyurethane or polyurethane-containing polymers have been used to fabricate a large number of implantable devices, including pacemaker leads, artificial hearts, heart valves, stent coverings, artificial tendons, arteries and veins. Formulations for delivery of active agents using polyurethane implantable devices, however, require a liquid medium or carrier for the diffusion of the drug at a zero order rate.

### SUMMARY

Described herein are methods and compositions based on the unexpected discovery that solid formulations comprising naltrexone can be used at the core of a polyurethane implantable device such that naltrexone is released in a controlled-release, zero-order manner from the implantable device. The polyurethane coating can be selected based on various physical parameters of naltrexone and the coating, and then the release rate of naltrexone from the implantable device can be optimized to a clinically relevant release rate based on clinical and/or in vitro trials.

One embodiment is directed to a method for delivering a formulation comprising an effective amount of naltrexone to a subject, comprising: implanting an implantable device into the subject, wherein the implantable device comprises naltrexone or a formulation thereof substantially surrounded by a polyurethane-based polymer. In a particular embodiment, the polyurethane-based polymer is formed from one or more polyols, wherein the general polyol structure is selected from the group consisting of:

—[O—(CH₂)ₙ]ₓ—O—;

O—(CH₂—CH₂—CH₂—CH₂)ₓ—O—; and

O—[(CH₂)₆—CO₃]ₙ—(CH₂)—O—.

In a particular embodiment, the polyol comprises —[O—(CH₂)ₙ]ₓ—O—, and wherein the polyurethane-based polymer has an equilibrium water content of between about 5% and about 200%. In a particular embodiment, the polyurethane-based polymer has an equilibrium water content of at least about 31%. In a particular embodiment, naltrexone is released at a zero-order rate of about 883 μg/day per square centimeter of the surface area of the implantable device. In a particular embodiment, the polyol comprises O—(CH₂—CH₂—CH₂—CH₂)ₓ—O—, and wherein the polyurethane-base polymer has a flex modulus of between about 1000 and about 92,000 psi. In a particular embodiment, the polyurethane-based polymer has a flex modulus of about 10,000 psi. In a particular embodiment, naltrexone is released at a zero-order rate of about 23 μg/day per square centimeter of the surface area of the implantable device. In a particular embodiment, the polyol comprises O—[(CH₂)₆—CO₃]ₙ—(CH₂)—O—, and wherein the polyurethane-based polymer has a flex modulus of between about 620 and about 92,000 psi. In a particular embodiment, the polyurethane-based polymer has a flex modulus of about 4,500 psi. In a particular embodiment, naltrexone is released at a zero-order rate of about 5.5 μg/day per square centimeter of the surface area of the implantable device.

One embodiment is directed to a drug delivery device for the controlled release of naltrexone over an extended period of time to produce local or systemic pharmacological effects, comprising: a) a polyurethane-based polymer formed to define a hollow space; and b) a solid drug formulation comprising a formulation comprising naltrexone and optionally one or more pharmaceutically acceptable carriers, wherein the solid drug formulation is contained in the hollow space, and wherein the device provides a desired release rate of naltrexone from the device after implantation. In a particular embodiment, the drug delivery device is conditioned and primed under conditions chosen to be consistent with the water solubility characteristics of the at least one active agent. In a particular embodiment, the pharmaceutically acceptable carrier is stearic acid. In a particular embodiment, the polyurethane-based polymer is formed from one or more polyols, wherein the general polyol structure is selected from the group consisting of:

—[O—(CH₂)ₙ]ₓ—O—;

O—(CH₂—CH₂—CH₂—CH₂)ₓ—O—; and

O—[(CH₂)₆—CO₃]ₙ—(CH₂)—O—.

In a particular embodiment, the polyol comprises —[O—(CH₂)ₙ]ₓ—O—, and wherein the polyurethane-based polymer has an equilibrium water content of between about 5% and about 200%. In a particular embodiment, the polyurethane-based polymer has an equilibrium water content of at least about 31%. In a particular embodiment, naltrexone is released at a zero-order rate of about 883 μg/day per square centimeter of the surface area of the implantable device. In a particular embodiment, the polyol comprises O—(CH₂—CH₂—CH₂—CH₂)ₓ—O—, and wherein the polyurethane-base polymer has a flex modulus of between about 1000 and about 92,000 psi. In a particular embodiment, the polyurethane-based polymer has a flex modulus of about 10,000 psi. In a particular embodiment, naltrexone is released at a zero-order rate of about 23 μg/day per square centimeter of the surface area of the implantable device. In a particular embodiment, the polyol comprises O—[(CH₂)₆—CO₃]n—(CH₂)—O—, and wherein the polyurethane-based polymer has a flex modulus of between about 620 and about 92,000 psi. In a particular embodiment, the polyurethane-based polymer has a flex modulus of about 4,500 psi. In a particular embodiment, naltrexone is released at a zero-order rate of about 5.5 μg/day per square centimeter of the surface area of the implantable device. In a particular embodiment, the appropriate conditioning and priming parameters can be selected to establish the desired delivery rates of the at least one active agent, including the manipulation of time, temperature, conditioning medium, priming medium or combinations thereof.

### BRIEF DESCRIPTION OF THE DRAWINGS

FIG. 1 is a side view of an implant with two open ends.

FIG. 2 is a side view of pre-fabricated end plugs used to plug the implants.

FIG. 3 is a side view of an implant with one open end.

FIG. 4 is a graph of the elution rate of naltrexone from an implant.

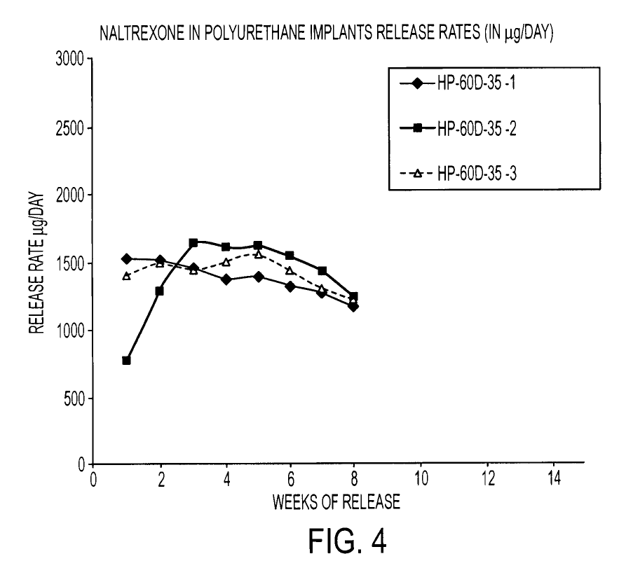

FIG. 5 is a graph of the elution rate of naltrexone from polyurethane implants.

FIG. 6 is a graph showing the direct correlation between in vitro implant release rate (mcg/day) and in vivo concentration (pg/mL) of naltrexone in the blood.

### DETAILED DESCRIPTION OF PREFERRED EMBODIMENTS

To take the advantage of the excellent properties of polyurethane-based polymers, the present invention is directed to the use of polyurethane-based polymers as drug delivery devices for releasing drugs at controlled rates for an extended period of time to produce local or systemic pharmacological effects. The drug delivery device can comprise a cylindrically shaped reservoir surrounded by polyurethane-based polymer that controls the delivery rate of the drug inside the reservoir. The reservoir contains a formulation, e.g., a solid formulation, comprising one or more active ingredients and, optionally, pharmaceutically acceptable carriers. The carriers are formulated to facilitate the diffusion of the active ingredients through the polymer and to ensure the stability of the drugs inside the reservoir.

A polyurethane is any polymer consisting of a chain of organic units joined by urethane links. Polyurethane polymers are formed by reacting a monomer containing at least two isocyanate functional groups with another monomer containing at least two alcohol groups in the presence of a catalyst. Polyurethane formulations cover an extremely wide range of stiffness, hardness, and densities.

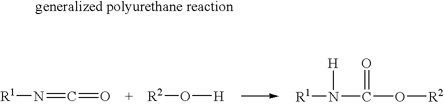

Polyurethanes are in the class of compounds called “reaction polymers,” which include epoxies, unsaturated polyesters and phenolics. A urethane linkage is produced by reacting an isocyanate group, —N═C═O with a hydroxyl (alcohol) group, —OH. Polyurethanes are produced by the polyaddition reaction of a polyisocyanate with a polyalcohol (polyol) in the presence of a catalyst and other additives. In this case, a polyisocyanate is a molecule with two or more isocyanate functional groups, R—(N═C═O)_{n≧2} and a polyol is a molecule with two or more hydroxyl functional groups, R′—(OH)_{n≧2}. The reaction product is a polymer containing the urethane linkage, —RNHCOOR′—. Isocyanates react with any molecule that contains an active hydrogen. Importantly, isocyanates react with water to form a urea linkage and carbon dioxide gas; they also react with polyetheramines to form polyureas.

Polyurethanes are produced commercially by reacting a liquid isocyanate with a liquid blend of polyols, catalyst, and other additives. These two components are referred to as a polyurethane system, or simply a system. The isocyanate is commonly referred to in North America as the “A-side” or just the “iso,” and represents the rigid backbone (or “hard segment”) of the system. The blend of polyols and other additives is commonly referred to as the “B-side” or as the “poly,” and represents the functional section (or “soft segment”) of the system. This mixture might also be called a “resin” or “resin blend.” Resin blend additives can include chain extenders, cross linkers, surfactants, flame retardants, blowing agents, pigments and fillers. In drug delivery applications, the “soft segments” represent the section of the polymer that imparts the characteristics that determine the diffusivity of an active pharmaceutical ingredient (API) through that polymer.

The elastomeric properties of these materials are derived from the phase separation of the hard and soft copolymer segments of the polymer, such that the urethane hard segment domains serve as cross-links between the amorphous polyether (or polyester) soft segment domains. This phase separation occurs because the mainly non-polar, low-melting soft segments are incompatible with the polar, high-melting hard segments. The soft segments, which are formed from high molecular weight polyols, are mobile and are normally present in coiled formation, while the hard segments, which are formed from the isocyanate and chain extenders, are stiff and immobile. Because the hard segments are covalently coupled to the soft segments, they inhibit plastic flow of the polymer chains, thus creating elastomeric resiliency. Upon mechanical deformation, a portion of the soft segments are stressed by uncoiling, and the hard segments become aligned in the stress direction. This reorientation of the hard segments and consequent powerful hydrogen-bonding contributes to high tensile strength, elongation, and tear resistance values.

The polymerization reaction is catalyzed by tertiary amines, such as, for example, dimethylcyclohexylamine, and organometallic compounds, such as, for example, dibutyltin dilaurate or bismuth octanoate. Furthermore, catalysts can be chosen based on whether they favor the urethane (gel) reaction, such as, for example, 1,4-diazabicyclo[2.2.2]octane (also called DABCO or TEDA), or the urea (blow) reaction, such as bis-(2-dimethylaminoethyl)ether, or specifically drive the isocyanate trimerization reaction, such as potassium octoate.

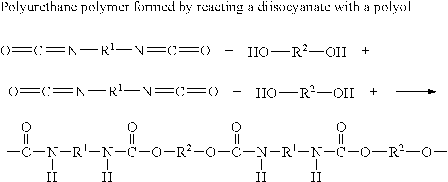

Isocyanates with two or more functional groups are required for the formation of polyurethane polymers. Volume wise, aromatic isocyanates account for the vast majority of global diisocyanate production. Aliphatic and cycloaliphatic isocyanates are also important building blocks for polyurethane materials, but in much smaller volumes. There are a number of reasons for this. First, the aromatically linked isocyanate group is much more reactive than the aliphatic one. Second, aromatic isocyanates are more economical to use. Aliphatic isocyanates are used only if special properties are required for the final product. Light stable coatings and elastomers, for example, can only be obtained with aliphatic isocyanates. Aliphatic isocyanates also are favored in the production of polyurethane biomaterials due to their inherent stability and elastic properties.

Examples of aliphatic and cycloaliphatic isocyanates include, for example, 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane (H₁₂MDI). They are used to produce light stable, non-yellowing polyurethane coatings and elastomers. H₁₂MDI prepolymers are used to produce high performance coatings and elastomers with optical clarity and hydrolysis resistance. Tecoflex®, Tecophilic® and Carbothane® polyurethanes are all produced from H₁₂MDI prepolymers.

Polyols are higher molecular weight materials manufactured from an initiator and monomeric building blocks, and, where incorporated into polyurethane systems, represent the “soft segments” of the polymer. They are most easily classified as polyether polyols, which are made by the reaction of epoxides (oxiranes) with an active hydrogen containing starter compounds, or polyester polyols, which are made by the polycondensation of multifunctional carboxylic acids and hydroxyl compounds.

Tecoflex® polyurethanes and Tecophilic® polyurethanes are cycloaliphatic polymers and are of the types produced from polyether-based polyols. For the Tecoflex® polyurethanes, the general structure of the polyol segment is represented as,

O—(CH₂—CH₂—CH₂—CH₂)ₓ—O—

whereby an increase in “x” represents a increase in flexibility (decreased “Flex Modulus”; “FM”), yielding FM ranging from about 1000-92,000 psi. From the standpoint of drug release from these materials, the release of a relatively hydrophobic API decreases as the FM increases.

For the Tecophilic® (hydrophilic) polyurethanes, the general structure of the polyol segment is represented as,

—[O—(CH₂)ₙ]ₓ—O—

whereby increases in “n” and “x” represent variations in hydrophilicity, and yield equilibrium water contents (% EWC) ranging from about 5%-43%. From the standpoint of drug release from these materials, the release of a relatively hydrophilic API increases as the % EWC increases.

Specialty polyols include, for example, polycarbonate polyols, polycaprolactone polyols, polybutadiene polyols, and polysulfide polyols.

Carbothane® polyurethanes are cycloaliphatic polymers and are of the types produced from polycarbonate-based polyols. The general structure of the polyol segment is represented as,

O—[(CH₂)₆—CO₃]ₙ—(CH₂)—O—

whereby an increase in “n” represents a increase in flexibility (decreased FM), yielding FM ranging from about 620-92,000 psi. From the standpoint of drug release from these materials, the release of a relatively hydrophobic API will decrease as the FM increases.

Chain extenders and cross linkers are low molecular weight hydroxyl- and amine-terminated compounds that play an important role in the polymer morphology of polyurethane fibers, elastomers, adhesives and certain integral skin and microcellular foams. Examples of chain extenders include, for example, ethylene glycol, 1,4-butanediol (1,4-BDO or BDO), 1,6-hexanediol, cyclohexane dimethanol and hydroquinone bis(2-hydroxyethyl)ether (HQEE). All of these glycols form polyurethanes that phase separate well, form well-defined hard segment domains, and are melt processable. They are all suitable for thermoplastic polyurethanes with the exception of ethylene glycol, since its derived bis-phenyl urethane undergoes unfavorable degradation at high hard segment levels. Tecophilic®, Tecoflex® and Carbothane® polyurethanes all incorporate the use of 1,4-butanediol as the chain extender.

The current invention provides a drug delivery device that can achieve the following objectives: a controlled-release rate (e.g., zero-order release rate) to maximize therapeutic effects and minimize unwanted side effects, an easy way to retrieve the device if it is necessary to end the treatment, an increase in bioavailability with less variation in absorption and no first pass metabolism.

The release rate of the drug is governed by the Fick's Law of Diffusion as applied to a cylindrically shaped reservoir device (cartridge). The following equation describes the relationship between different parameters:

$$\frac{d M}{d t} = \frac{2 π hp Δ C}{ln ( {r}_{o} / {r}_{i} )}$$

where:
dM/dt: drug release rate;
h: length of filled portion of device;
ΔC: concentration gradient across the reservoir wall;
rₒ/rᵢ: ratio of outside to inside radii of device; and
p: permeability coefficient of the polymer used.

The permeability coefficient is primarily regulated by the hydrophilicity or hydrophobicity of the polymer, the structure of the polymer, and the interaction of drug and the polymer. Once the polymer and the active ingredient are selected, p is a constant, h, rₒ, and rᵢ are fixed and kept constant once the cylindrically shaped device is produced. ΔC is maintained constant.

To keep the geometry of the device as precise as possible, the device, e.g., a cylindrically shaped device, can be manufactured through precision extrusion or precision molding process for thermoplastic polyurethane polymers, and reaction injection molding or spin casting process for thermosetting polyurethane polymers.

The cartridge can be made with either one end closed or both ends open. The open end can be plugged with, for example, pre-manufactured end plug(s) to ensure a smooth end and a solid seal, or, in the case of thermoplastic polyurethanes, by using heat-sealing techniques known to those skilled in the art. The solid actives and carriers can be compressed into pellet form to maximize the loading of the actives.

To identify the location of the implant, radiopaque material can be incorporated into the delivery device by inserting it into the reservoir or by making it into end plug to be used to seal the cartridge.

Once the cartridges are sealed on both ends with the filled reservoir, they are optionally conditioned and primed for an appropriate period of time to ensure a constant delivery rate.

The conditioning of the drug delivery devices involves the loading of the actives (drug) into the polyurethane-based polymer that surrounds the reservoir. The priming is done to stop the loading of the drug into the polyurethane-based polymer and thus prevent loss of the active before the actual use of the implant. The conditions used for the conditioning and priming step depend on the active, the temperature and the medium in which they are carried out. The conditions for the conditioning and priming may be the same in some instances.

The conditioning and priming step in the process of the preparation of the drug delivery devices is done to obtain a determined rate of release of a specific drug. The conditioning and priming step of the implant containing a hydrophilic drug can be carried out in an aqueous medium, e.g., in a saline solution. The conditioning and priming step of a drug delivery device comprising a hydrophobic drug is usually carried out in a hydrophobic medium such as, for example, an oil-based medium. The conditioning and priming steps can be carried out by controlling three specific factors, namely the temperature, the medium and the period of time.

A person skilled in the art would understand that the conditioning and priming step of the drug delivery device is affected by the medium in which the device is placed. A hydrophilic drug can be conditioned and primed, for example, in an aqueous solution, e.g., in a saline solution. Naltrexone implants, for example, have been conditioned and primed in saline solution, more specifically, conditioned in saline solution of 0.9% sodium content and primed in saline solution of 1.8% sodium chloride content. The temperature used to condition and prime the drug delivery device can vary across a wide range of temperatures, e.g., about 37° C.

The time period used for the conditioning and priming of the drug delivery devices can vary from about a single day to several weeks depending on the release rate desired for the specific implant or drug. The desired release rate is determined by one of skill in the art with respect to the particular active agent used in the pellet formulation.

A person skilled in the art will understand the steps of conditioning and priming the implants are to optimize the rate of release of the drug contained within the implant. As such, a shorter time period spent on the conditioning and the priming of a drug delivery device results in a lower rate of release of the drug compared to a similar drug delivery device that has undergone a longer conditioning and priming step.

The temperature in the conditioning and priming step will also affect the rate of release in that a lower temperature results in a lower rate of release of the drug contained in the drug delivery device when compared to a similar drug delivery device that has undergone a treatment at a higher temperature.

Similarly, in the case of aqueous solutions, e.g., saline solutions, the sodium chloride content of the solution determines what type of rate of release will be obtained for the drug delivery device. More specifically, a lower content of sodium chloride results in a higher rate of release of drug when compared to a drug delivery device that has undergone a conditioning and priming step where the sodium chloride content was higher.

The same conditions apply for hydrophobic drugs where the main difference in the conditioning and priming step is that the conditioning and priming medium is a hydrophobic medium, more specifically an oil-based medium.

The delivery of naltrexone can be useful, for example, for the management of alcohol dependence and opioid dependence. Low dose naltrexone, where the drug is used in doses approximately one-tenth those used for drug/alcohol rehabilitation purposes, is used as a treatment for certain immunologically related disorders, including, for example, HIV/AIDS, multiple sclerosis (in particular, the primary progressive variant), Parkinson's disease, cancer, autoimmune diseases such as, for example, rheumatoid arthritis or ankylosing spondylitis, Crohn's disease, ulcerative colitis, and central nervous system disorders. Naltrexone can induce early morning erections in patients who suffer from psychogenic erectile dysfunction. Naltrexone has been shown to be effective in the reversal of sexual satiety and exhaustion in male rats. Naltrexone improves smoking cessation rates in women by fifty percent. Some self-injurious behaviors present in developmentally disabled and autistic people can sometimes be remedied with naltrexone. Naltrexone is an opioid receptor antagonist marketed in generic form as its hydrochloride salt, naltrexone hydrochloride, and marketed under the trade names Revia® and Depade®. Effective levels of naltrexone in the blood are known and established and can range, for example, about 0.1 to about 2 ng/ml, from about 0.2 to about 1 ng/ml or about 0.3 to about 0.8 ng/ml range. Implants with in vitro release rates of 220 μg/day, 440 μg/day and 880 μg/day led to in vivo blood concentrations of about 0.2 ng/ml, about 0.3 ng/ml and about 0.7 ng/ml, respectively, indicating a near linear relationship between in vitro implant release rate and in vivo concentration of naltrexone in the blood.

One of skill in the art would be able to tailor naltrexone release by altering a variety of implant factors. For example, as shown in the Examples, different classes of polyurethanes lead to different release rates of naltrexone. Additionally, within classes of polyurethanes, the EWC and/or flex modulus of the polyurethane can be varied to achieve different naltrexone release rates. Further still, one of skill in the art could vary the size of the implant to increase or decrease the surface area of the implant, thereby varying the release rate of naltrexone from the implant. Such alterations lead to release rates in the physiologically relevant range, e.g., of about 0.001 to about 3 mg/day, from about 0.1 to about 3 mg/day, from about 0.2 to about 2.5 mg/day, from about 0.5 to about 1.5 mg/day or at about 0.7 mg/day. Release rate from implants can also be varied, for example, by adjusting the amount and nature of excipients contained in the naltrexone formulation.

Implants that achieve physiological release rates of naltrexone can vary in size, depending on, for example, the nature of the polyurethane used. A cylindrical implant, for example, can have a range of internal diameters from about 1 mm to about 10 mm, from about 1.5 mm to about 5 mm, from about 1.8 mm to about 3.6 mm, about 3.6 mm or about 1.8 mm. An implant can range in length from about, for example, 5 mm to about 100 mm, from about 7.5 mm to about 50 mm, from about 10 mm to about 40 mm, from about 15 mm to about 30 mm, about 37 mm, about 40 mm or about 15.24 mm.

The current invention focuses on the application of polyurethane-based polymers, thermoplastics or thermosets, to the creation of implantable drug devices to deliver biologically active compounds at controlled rates for prolonged period of time. Polyurethane polymers can be made into, for example, cylindrical hollow tubes with one or two open ends through extrusion, (reaction) injection molding, compression molding, or spin-casting (see e.g., U.S. Pat. Nos. 5,266,325 and 5,292,515), depending on the type of polyurethane used.

Thermoplastic polyurethane can be processed through extrusion, injection molding or compression molding. Thermoset polyurethane can be processed through reaction injection molding, compression molding, or spin-casting. The dimensions of the cylindrical hollow tube should be as precise as possible.

Polyurethane-based polymers are synthesized from multi-functional polyols, isocyanates and chain extenders. The characteristics of each polyurethane can be attributed to its structure.

Thermoplastic polyurethanes are made of macrodiols, diisocyanates, and difunctional chain extenders (e.g., U.S. Pat. Nos. 4,523,005 and 5,254,662). Macrodiols make up the soft domains. Diisocyanates and chain extenders make up the hard domains. The hard domains serve as physical crosslinking sites for the polymers. Varying the ratio of these two domains can alter the physical characteristics of the polyurethanes, e.g., the flex modulus.

Thermoset polyurethanes can be made of multifunctional (greater than difunctional) polyols and/or isocyanates and/or chain extenders (e.g., U.S. Pat. Nos. 4,386,039 and 4,131,604). Thermoset polyurethanes can also be made by introducing unsaturated bonds in the polymer chains and appropriate crosslinkers and/or initiators to do the chemical crosslinking (e.g., U.S. Pat. No. 4,751,133). By controlling the amounts of crosslinking sites and how they are distributed, the release rates of the actives can be controlled.

Different functional groups can be introduced into the polyurethane polymer chains through the modification of the backbones of polyols depending on the properties desired. Where the device is used for the delivery of water soluble drugs, hydrophilic pendant groups such as ionic, carboxyl, ether, and hydroxyl groups are incorporated into the polyols to increase the hydrophilicity of the polymer (e.g., U.S. Pat. Nos. 4,743,673 and 5,354,835). Where the device is used for the delivery of hydrophobic drugs, hydrophobic pendant groups such as alkyl, siloxane groups are incorporated into the polyols to increase the hydrophobicity of the polymer (e.g., U.S. Pat. No. 6,313,254). The release rates of the actives can also be controlled by the hydrophilicity/hydrophobicity of the polyurethane polymers.

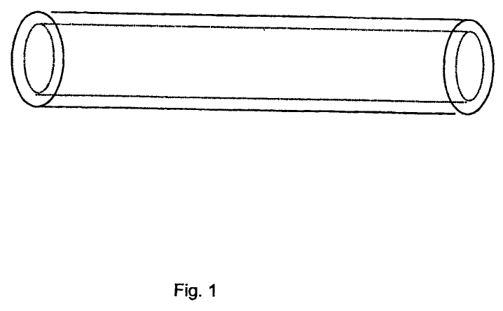

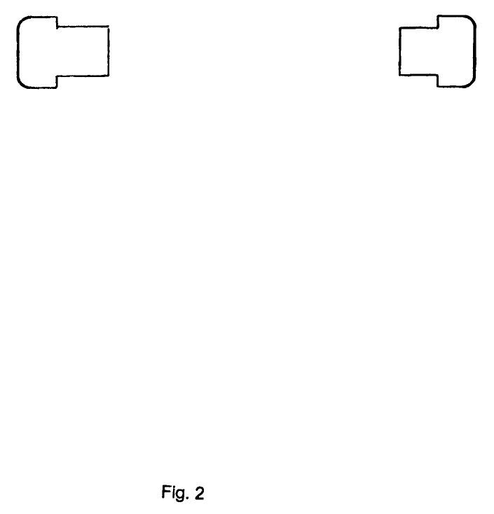

For thermoplastic polyurethanes, precision extrusion and injection molding are the preferred choices to produce two open-end hollow tubes (FIG. 1) with consistent physical dimensions. The reservoir can be loaded freely with appropriate formulations containing actives and carriers or filled with pre-fabricated pellets to maximize the loading of the actives. One open end needs to be sealed first before the loading of the formulation into the hollow tube. To seal the two open ends, two pre-fabricated end plugs (FIG. 2) can be used. The sealing step can be accomplished through the application of heat or solvent or any other means to seal the ends, preferably permanently.

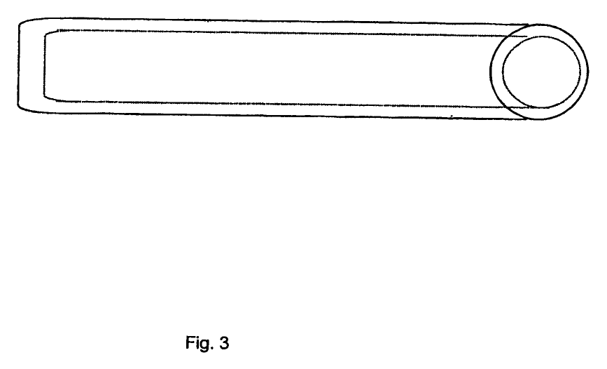

For thermoset polyurethanes, precision reaction injection molding or spin casting is the preferred choice depending on the curing mechanism. Reaction injection molding is used if the curing mechanism is carried out through heat and spin casting is used if the curing mechanism is carried out through light and/or heat. Hollow tubes with one open end (FIG. 3), for example, can be made by spin casting. Hollow tubes with two open ends, for example, can be made by reaction injection molding. The reservoir can be loaded in the same way as the thermoplastic polyurethanes.

To seal an open end, an appropriate light-initiated and/or heat-initiated thermoset polyurethane formulation can be used to fill the open end, and this is cured with light and/or heat. A pre-fabricated end plug, for example, can also be used to seal the open end by applying an appropriate light-initiated and/or heat-initiated thermoset polyurethane formulation on to the interface between the pre-fabricated end plug and the open end, and curing it with the light and/or heat or any other means to seal the ends, preferably permanently.

The final process involves the conditioning and priming of the implants to achieve the delivery rates required for the actives. Depending upon the types of active ingredient, hydrophilic or hydrophobic, the appropriate conditioning and priming media is chosen. Water-based media are preferred for hydrophilic actives, and oil-based media are preferred for hydrophobic actives.

As a person skilled in the art would readily know many changes can be made to the preferred embodiments of the invention without departing from the scope thereof. It is intended that all matter contained herein be considered illustrative of the invention and not it a limiting sense.

### EXEMPLIFICATION

### Example 1

Tecophilic® polyurethane polymer tubes are supplied by Thermedics Polymer Products and manufactured through a precision extrusion process. Tecophilic® polyurethane is a family of aliphatic polyether-based thermoplastic polyurethane that can be formulated to different equilibrium water contents (EWC) of up to 150% of the weight of dry resin. Extrusion grade formulations are designed to provide maximum physical properties of thermoformed tubing or other components. An exemplary tube and end cap structures are depicted in FIGS. 1-3.

The physical data for the polymers is provided below as made available by Thermedics Polymer Product (tests conducted as outlined by American Society for Testing and Materials (ASTM), Table 1).

TABLE 1
Tecophilic ® Typical Physical Test Data

| | | HP-60D- | HP-60D- | HP-60D- | HP-93A- |
|---|---|---|---|---|---|
| | ASTM | 20 | 35 | 60 | 100 |
| Durometer | D2240 | 43D | 42D | 41D | 83A |
| (Shore | | | | | |
| Hardness) | | | | | |
| Spec Gravity | D792 | 1.12 | 1.12 | 1.15 | 1.13 |
| Flex Modulus | D790 | 4,300 | 4,000 | 4,000 | 2,900 |
| (psi) | | | | | |
| Ultimate | D412 | 8,900 | 7,800 | 8,300 | 2,200 |
| Tensile Dry | | | | | |
| (psi) | | | | | |
| Ultimate | D412 | 5,100 | 4,900 | 3,100 | 1,400 |
| Tensile Wet | | | | | |
| (psi) | | | | | |
| Elongation Dry | D412 | 430 | 450 | 500 | 1,040 |
| (%) | | | | | |
| Elongation Wet | D412 | 390 | 390 | 300 | 620 |
| (%) | | | | | |

HP-60D-20 is extruded to tubes with thickness of 0.30 mm with inside diameter of 1.75 mm. The tubes are then cut into 25 mm in length. One side of the tube is sealed with heat using a heat sealer. The sealing time is less than one minute.

### Example 2

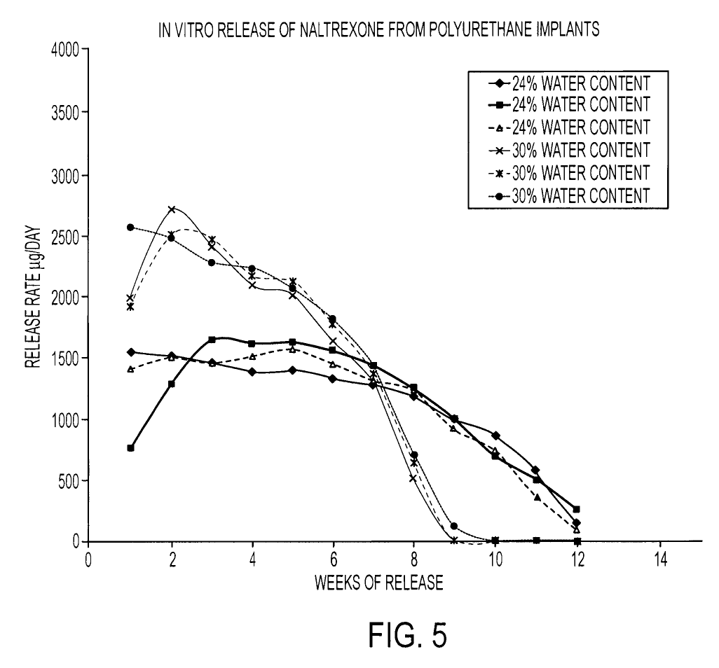

HP-60D-35 is extruded to tubes with thickness of 0.30 mm with inside diameter of 1.75 mm. The tubes are then cut into 32 mm in length. One side of the tube is sealed with heat using a heat sealer. The sealing time is less than one minute. Six pellets of naltrexone are loaded into the tubes and both open sides of the tubes are sealed with heat. Each pellet weighs approximately 15.0 mg for a total of 91 mg. The second end open of the tube is sealed with heat in the same way as for the first end. The loaded implant is then conditioned and primed. The conditioning takes place at room temperature in a 0.9% saline solution for one week. Upon completion of the conditioning, the implant undergoes priming. The priming takes place at room temperatures in a 1.8% saline solution for one week. Each implant is tested in vitro in a medium selected to mimic the pH found in the human body. The temperature of the selected medium was kept at approximately 37° C. during the testing. The release rates are shown on FIG. 5 and Table 2

TABLE 2
Naltrexone Elution Rates

| WEEKS OF | HP-60D-35-1 | HP-60D-35-2 | HP-60D-35-3 |
|---|---|---|---|
| RELEASE | (μg/day) | (μg/day) | (μg/day) |
| 0 | | | |
| 1 | 1529.26 | 767.38 | 1400.95 |
| 2 | 1511.77 | 1280.03 | 1498.86 |
| 3 | 1456.01 | 1635.97 | 1449.49 |
| 4 | 1378.27 | 1607.13 | 1500.42 |
| 5 | 1393.05 | 1614.52 | 1558.37 |
| 6 | 1321.71 | 1550.39 | 1436.03 |
| 7 | 1273.07 | 1424.24 | 1300.73 |
| 8 | 1172.82 | 1246.48 | 1221.57 |

### Example 3

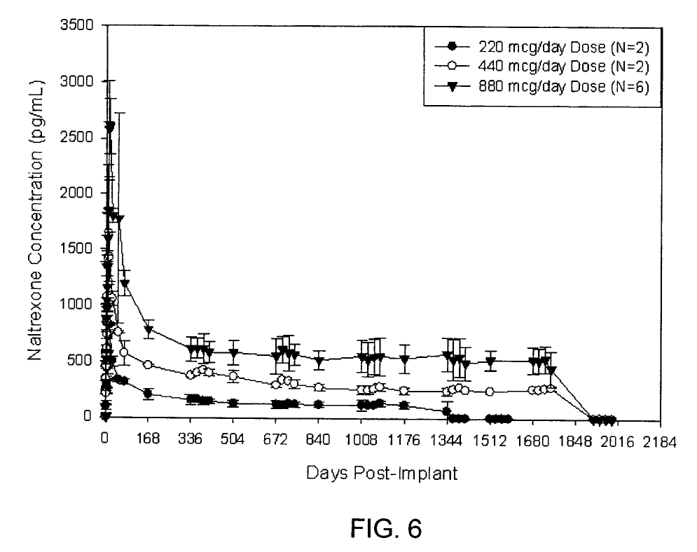

In FIG. 6 there is a comparison of the release rates of naltrexone in vitro using two grades of polymer at two different water contents. Three runs were carried out and analyzed where the polymer of the implant had a water content of 24% and three runs were carried out where the polymer of the implant had a water content of 30%. The release rates were plotted against time. The polymer used for the runs at 24% water content was Tecophilic® HP-60-D35 from Thermedics. The polymer used for the runs at 30% water content was Tecophilic® HP-60-D60 from Thermedics. The data obtained in this example demonstrate the good reproducibility of the implants as prepared according to the present invention.

### Example 4

Tables 3A-C show release rates of naltrexone from different classes of polyurethane compounds (Tecophilic®, Tecoflex® and Carbothane®). The release rates have been normalized to surface area of the implant, thereby adjusting for slight differences in the size of the various implantable devices. Naltrexone is considered to be hydrophobic (not very water soluble), as indicated by the Log P value; for the purposes of the data provided, a Log P value of greater than about 2.0 is considered to be not readily soluble in aqueous solution. The polyurethanes were selected to have varying affinities for water soluble active agents and varying flexibility (as indicated by the variation in flex modulus).

For applications of the polyurethanes useful for the devices and methods described herein, the polyurethane exhibits physical properties suitable for the specific active agent to be delivered. Polyurethanes are available or can be prepared, for example, with a range of EWCs or flex moduli (Table 3). Tables 3A-C show normalized release rates for various active ingredients from polyurethane compounds. Tables 3D-F show the non-normalized release rates for the same active ingredients, together with implant composition.

TABLE 3A

| | | Polyurethane Type | | | | |
|---|---|---|---|---|---|---|
| | | Tecophilic | | | | |
| | | Polyurethane Grade | | | | |
| | | HP-60D-60 | HP-60D-35 | HP-60D-20 | HP-60D-10 | HP-60D-05 |
| | Relative Water | % EWC/Flex Modulus | | | | |
| Active | Solubility | 31% EWC | 24% EWC | 15% EWC | 8.7% EWC | 5.5% EWC |
| Naltrexone | Log P = 2.07 | 883 μg/day/cm² | 704 μg/day/cm² | 263 μg/day/cm² | 127 μg/day/cm² | 12.7 μg/day/cm² |
| Base | | 10% CC, 2% SA + | 2% SA | 10% CC, 2% SA, | 10% CC, 2% SA + | 10% CC, 2% SA, |
| (M.W. 285) | | PEG 400, 79.2 mg API | 91.3 mg API | 193.6 mg API | PEG 400, 197.1 mg API | 144.6 mg API |

TABLE 3B

| | | Polyurethane Type | | |
|---|---|---|---|---|
| | | Tecoflex | | |
| | | Polyurethane Grade | | |
| | Relative | EG-85A | EG 100A | EG-65D |
| | Water | % EWC/Flex Modulus | | |
| Active | Solubility | F.M.: 2,300 | F.M.: 10,000 | F.M.: 37,000 |
| Naltrexone | Log P = 2.07 | — | 23 μg/day/cm² | — |
| Base | | | 10% CC, 2% | |
| (M.W. 285) | | | SA, 75.5 mg | |
| | | | API | |

TABLE 3C

| | | Polyurethane Type | |
|---|---|---|---|
| | | Carbothane | |
| | | Polyurethane Grade | |
| | | PC-3575A | PC-3595A |
| | Relative Water | % EWC/Flex Modulus | |
| Active | Solubility | F.M.: 620 | F.M.: 4,500 |
| Naltrexone Base | Log P = 2.07 | — | 5.5 μg/day/cm² |
| (M.W. 285) | | | 10% CC, 2% SA, |
| | | | 189.2 mg API |

TABLE 3D

| | | Polyurethane | | | | |
|---|---|---|---|---|---|---|
| | | Tecophilic | | | | |
| | | Grade | | | | |
| | Relative | HP-60D-60 | HP-60D-35 | HP-60D-20 | HP-60D-10 | HP-60D-05 |
| | Water | % EWC | | | | |
| Active | Solubility | 31% EWC | 24% EWC | 15% EWC | 8.7% EWC | 5.5% EWC |
| Naltrexone | Log P = 2.07 | 2200 μg/day | 1500 μg/day | 1000 μg/day | 500 μg/day | 50 μg/day |
| Base | | ID: 1.80 mm | ID: 1.80 mm | ID: 2.87 mm | ID: 3.05 mm | ID: 3.05 mm |
| (M.W. 285) | | Wall: 0.30 mm | Wall: 0.30 mm | Wall: 0.38 mm | Wall: 0.30 mm | Wall: 0.30 mm |
| | | L: 37.8 mm | L: 32.3 mm | L: 37.2 mm | L: 37.3 mm | L: 37.4 mm |
| | | 2.492 cm² | 2.130 cm² | 3.796 cm² | 3.924 cm² | 3.934 cm² |

TABLE 3E

| | | Polyurethane Type | | |
|---|---|---|---|---|
| | | Tecoflex | | |
| | | Polyurethane Grade | | |
| | Relative | EG-85A | EG 100A | EG-65D |
| | Water | Flex Modulus | | |
| Active | Solubility | F.M.: 2,300 | F.M.: 10,000 | F.M.: 37,000 |
| Naltrexone | Log P = 2.07 | — | 55 μg/day | — |
| Base | | | ID: 1.85 mm | |
| (M.W. 285) | | | Wall: 0.20 mm | |
| | | | L: 37.49 mm | |
| | | | 2.413 cm² | |

TABLE 3F

Polyurethane Type
Carbothane
Polyurethane Grade

| | | |
|---|---|---|
| Relative | PC-3575A | PC-3595A |

| | |
|---|---|
| Water | Flex Modulus |

| Active | Solubility | F.M.: 620 | F.M.: 4,500 |
|---|---|---|---|
| Naltrexone Base | Log P = 2.07 | — | 25 μg/day |
| (M.W. 285) | | | ID: 3.63 mm |
| | | | Wall: 0.18 mm |
| | | | L: 38.19 mm |
| | | | 4.569 cm² |

The solubility of an active agent in an aqueous environment can be measured and predicted based on its partition coefficient (defined as the ratio of concentration of compound in aqueous phase to the concentration in an immiscible solvent). The partition coefficient (P) is a measure of how well a substance partitions between a lipid (oil) and water. The measure of solubility based on P is often given as Log P. In general, solubility is determined by Log P and melting point (which is affected by the size and structure of the compounds). Typically, the lower the Log P value, the more soluble the compound is in water. It is possible, however, to have compounds with high Log P values that are still soluble on account of, for example, their low melting point. It is similarly possible to have a low Log P compound with a high melting point, which is very insoluble.

The flex modulus for a given polyurethane is the ratio of stress to strain. It is a measure of the “stiffness” of a compound. This stiffness is typically expressed in Pascals (Pa) or as pounds per square inch (psi).

The elution rate of an active agent from a polyurethane compound can vary on a variety of factors including, for example, the relative hydrophobicity/hydrophilicity of the polyurethane (as indicated, for example, by logP), the relative “stiffness” of the polyurethane (as indicated, for example, by the flex modulus), and/or the molecular weight of the active agent to be released.

### EQUIVALENTS

The present disclosure is not to be limited in terms of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

While various aspects and embodiments have been disclosed herein, other aspects and embodiments will be apparent to those skilled in the art. All references cited herein are incorporated by reference in their entireties.

What is claimed is:

1. A method for delivering a formulation comprising an effective amount of naltrexone to a subject, comprising: implanting an implantable device into the subject, wherein the implantable device comprises naltrexone or a formulation thereof substantially surrounded by a polyurethane-based polymer.

2. The method of claim 1, wherein the polyurethane-based polymer is formed from one or more polyols, wherein the general polyol structure is selected from the group consisting of:
—[O—(CH₂)ₙ]ₓ—O—;
O—(CH₂—CH₂—CH₂—CH₂)ₓ—O—; and
O—[(CH₂)₆—CO₃]ₙ—(CH₂)—O—.

3. The method of claim 2, wherein the polyol comprises —[O—(CH₂)ₙ]ₓ—O—, and wherein the polyurethane-based polymer has an equilibrium water content of between about 5% and about 200%.

4. The method of claim 3, wherein the polyurethane-based polymer has an equilibrium water content of at least about 31%.

5. The method of claim 2, wherein naltrexone is released at a zero-order rate of about 883 μg/day per square centimeter of the surface area of the implantable device.

6. The method of claim 2, wherein the polyol comprises O—(CH₂—CH₂—CH₂—CH₂)ₓ—O—, and wherein the polyurethane-base polymer has a flex modulus of between about 1,000 and about 92,000 psi.

7. The method of claim 6, wherein the polyurethane-based polymer has a flex modulus of about 10,000 psi.

8. The method of claim 6, wherein naltrexone is released at a zero-order rate of about 23 μg/day per square centimeter of the surface area of the implantable device.

9. The method of claim 2, wherein the polyol comprises O—[(CH₂)₆—CO₃]ₙ—(CH₂)—O—, and wherein the polyurethane-based polymer has a flex modulus of between about 620 and about 92,000 psi.

10. The method of claim 9, wherein the polyurethane-based polymer has a flex modulus of about 4,500 psi.

11. The method of claim 9, wherein naltrexone is released at a zero-order rate of about 5.5 μg/day per square centimeter of the surface area of the implantable device.

12. A drug delivery device for the controlled release of naltrexone over an extended period of time to produce local or systemic pharmacological effects, comprising:
a) a polyurethane-based polymer formed to define a hollow space; and
b) a solid drug formulation comprising a formulation comprising naltrexone and optionally one or more pharmaceutically acceptable carriers,
wherein the solid drug formulation is contained in the hollow space, and wherein the device provides a desired release rate of naltrexone from the device after implantation.

13. The drug delivery device of claim 12, wherein the drug delivery device is conditioned and primed under conditions chosen to be consistent with the water solubility characteristics of the at least one active agent.

14. The drug delivery device of claim 13, wherein the pharmaceutically acceptable carrier is stearic acid.

15. The drug delivery device of claim 8, wherein the polyurethane-based polymer is formed from one or more polyols, wherein the general polyol structure is selected from the group consisting of:
—[O—(CH₂)ₙ]ₓ—O—;
O—(CH₂—CH₂—CH₂—CH₂)ₓ—O—; and
O—[(CH₂)₆—CO₃]ₙ—(CH₂)—O—.

16. The method of claim 15, wherein the polyol comprises —[O—(CH₂)ₙ]ₓ—O—, and wherein the polyurethane-based polymer has an equilibrium water content of between about 5% and about 200%.

17. The method of claim 16, wherein the polyurethane-based polymer has an equilibrium water content of at least about 31%.

18. The method of claim 15, wherein naltrexone is released at a zero-order rate of about 883 μg/day per square centimeter of the surface area of the implantable device.

19. The method of claim 15, wherein the polyol comprises O—(CH₂—CH₂—CH₂—CH₂)ₓ—O—, and wherein the polyurethane-base polymer has a flex modulus of between about 1,000 and about 92,000 psi.

20. The method of claim 19, wherein the polyurethane-based polymer has a flex modulus of about 10,000 psi.

21. The method of claim 19, wherein naltrexone is released at a zero-order rate of about 23 μg/day per square centimeter of the surface area of the implantable device.

22. The method of claim 15, wherein the polyol comprises O—[(CH₂)₆—CO₃]n—(CH₂)—O—, and wherein the polyurethane-based polymer has a flex modulus of between about 620 and about 92,000 psi.

23. The method of claim 22, wherein the polyurethane-based polymer has a flex modulus of about 4,500 psi.

24. The method of claim 22, wherein naltrexone is released at a zero-order rate of about 5.5 μg/day per square centimeter of the surface area of the implantable device.

25. The drug delivery device of claim 12, wherein the appropriate conditioning and priming parameters can be selected to establish the desired delivery rates of the at least one active agent, including the manipulation of time, temperature, conditioning medium, priming medium or combinations thereof.
